(19) United States
(12) Patent Application Publication
(10) Pub. No.: US 2026/0053634 A1
(43) Pub. Date: Feb. 26, 2026

(54) Adjustable Spinal Implants, Associated Instruments and Methods

(71) Applicant: Blue Ocean Spine GmbH, Tuttlingen (DE)

(72) Inventors: Guntmar Eisen, Tuttlingen (DE); Markus Salvermoser, Tuttlingen-Möhringen (DE); Jacob Richter, Tuttlingen (DE)

(21) Appl. No.: 19/371,739

(22) Filed: Oct. 28, 2025

Related U.S. Application Data
(63) Continuation of application No. 17/865,771, filed on Jul. 15, 2022, now Pat. No. 12,453,640.
(60) Provisional application No. 63/222,482, filed on Jul. 16, 2021.
(60) Provisional application No. 63/222,498, filed on Jul. 16, 2021.
(60) Provisional application No. 63/222,506, filed on Jul. 16, 2021.

(30) Foreign Application Priority Data
Jul. 16, 2021 (EP) 21186249.5
Jul. 16, 2021 (EP) 21186250.3
Jul. 16, 2021 (EP) 21186251.1
Jul. 16, 2021 (EP) 21186272.7

(51) Int. Cl. A61F 2/44; A61F 2/30; A61F 2/46

(52) U.S. Cl. CPC A61F 2/4455; A61F 2/4611; A61F 2002/30405; A61F 2002/3054; A61F 2002/30579; A61F 2002/30593; A61F 2002/30772; A61F 2002/4627

(57) ABSTRACT

The present disclosure provides adjustable spinal devices, instruments for implanting the spinal devices, methods for adjusting the height and lordosis angles of the spinal devices and methods for implanting such devices. An adjustable spinal fusion device includes an upper plate component having an outer surface for placement against an endplate of a vertebral body and a lower plate component having an outer surface for placement against an endplate of a vertebral body. The device further includes a first translation member configured to move longitudinally relative to the upper and lower plates to adjust a distance between the upper and lower plates and a second translation member configured to move longitudinally relative to the upper and lower plates to adjust an angle between the upper and lower plates.

### CROSS-REFERENCE TO RELATED APPLICATIONS

This application is a continuation of U.S. patent application Ser. No. 17/865,771, filed Jul. 15, 2022, which claims the benefit of U.S. Provisional Application Serial Nos. 63/222,482, 63/222,498 and 63/222,506, all of which were filed Jul. 16, 2021, and European Patent Application Nos. 21186249.5, 21186250.3 and 21186251.1, all of which were filed Jul. 16, 2021, the complete disclosures of which are incorporated herein by reference in their entirety for all purposes.

### FIELD

The present disclosure relates to implantable devices for stabilizing and/or promoting the fusion of adjacent bony structures and, more particularly, to implantable spinal fusion cages that can adjust in height and angle to accommodate spacing constraints and/or address lordosis within an intervertebral space.

### BACKGROUND

Implantable spinal devices can be used to treat a variety of spinal disorders, including degenerative disc disease. For example, in one type of spinal disorder, the intervertebral disc has deteriorated or become damaged due to acute injury or trauma, disc disease or simply the natural aging process. The standard treatment today may involve surgical removal of a portion, or all, of the diseased or damaged intervertebral disc in a process known as a partial or total discectomy, respectively. The discectomy is often followed by the insertion of a cage or spacer to stabilize this weakened or damaged spinal region. This cage or spacer serves to reduce or inhibit mobility in the treated area, in order to avoid further progression of the damage and/or to reduce or alleviate pain caused by the damage or injury. Moreover, these types of cages or spacers serve as mechanical or structural scaffolds to restore and maintain normal disc height, and in some cases, can also provide a space for inserting bone graft material to promote bony fusion between the adjacent vertebrae.

One of the current challenges of these types of procedures is the very limited working space afforded the surgeon to manipulate and insert the cage into the intervertebral area to be treated. Access to the intervertebral space requires navigation around retracted adjacent vessels and tissues such as the aorta, vena cava, dura and nerve roots, leaving a very narrow pathway for access. The opening to the intradiscal space itself is also relatively small. Hence, there are physical limitations on the actual size of the cage that can be inserted without significantly disrupting the surrounding tissue or the vertebral bodies themselves.

Further complicating the issue is the fact that the vertebral bodies are not positioned parallel to one another in a normal spine. There is a natural curvature to the spine due to the angular relationship of the vertebral bodies relative to one another. The ideal cage must be able to accommodate this angular relationship of the vertebral bodies, or else the cage will not sit properly when inside the intervertebral space. An improperly fitted cage would either become dislodged or migrate out of position, and lose effectiveness over time, or worse, further damage the already weakened area.

Another problem is that, In order to insert the cage between the adjacent vertebra, at least a portion, if not all, of the intervertebral disc is removed to make room for the cage. The removal of the entire disc or disc portion disrupts the normal lordotic or kyphotic curvature of the spine. Traditional fusion cages do not attempt to correct this curvature, and over time as the vertebrae settle around the implanted cages, kyphotic deformity results.

It is therefore desirable to provide implantable spinal devices that have the ability to maintain and restore the normal anatomy of the fused spine segment. It is particularly desirable to provide interbody cages or spacers that not only have the mechanical strength or structural integrity to restore disc height or vertebral alignment to the spinal segment to be treated, but also can easily pass through the narrow access pathway into the intervertebral space, and accommodate the angular constraints of this space and/or correct the lordotic or kyphotic curvature created by removal of the disc.

### SUMMARY

The present disclosure provides spinal systems, implantable spinal devices and instruments for implanting the spinal systems or devices. The present disclosure further provides methods for adjusting the height and lordosis angles of the spinal devices and methods for implanting such devices.

In one aspect, an adjustable spinal fusion device includes an upper plate having an outer surface for placement against a first vertebral body and a lower plate having an outer surface for placement against a second vertebral body. The device further includes a first translation member configured to move longitudinally relative to the upper and lower plates to adjust a distance between the upper and lower plates (i.e., the height of the implant); and a second translation member configured to move longitudinally relative to the upper and lower plates to adjust an angle between the upper and lower plates (i.e., the angle of lordosis of the implant). Thus, the device has a first configuration for advancing through a narrow access pathway into the intervertebral space, and a second configuration, wherein the device may be adjusted in height and/or angle to accommodate the angular constraints of this space and/or correct the lordotic or kyphotic curvature.

In embodiments, the upper and lower plates each have proximal and distal ends and first and second sides extending from the proximal end to the distal end. The second translation member is configured to adjust the angle between the first and second sides of the plates. This allows the upper plate to rotate relative to the lower plate around an axis substantially parallel to the longitudinal axis of the device.

In embodiments, the device comprises a hinge on one of the first and second sides for rotatably coupling the upper plate to the lower plate. The hinge may include a rod extending substantially longitudinally along one side of the plates. The upper plate may comprise one or more coupling elements, such as knuckles or the like, rotatably coupled around the rod for allowing rotation of the upper plate relative to the lower plate.

In embodiments, longitudinal movement of the first translation member causes longitudinal movement of the second translation member to adjust the distance between the upper and lower plates and longitudinal movement of the second translation member relative to the first translation member adjusts the angle between the upper and lower plates. This allows for independent adjustment of the angle and/or height of the device.

In embodiments, the first translation member comprises a first bore with a mating feature for cooperating with a first actuator shaft of a surgical instrument to move the first translation member in the longitudinal direction. The second translation member comprises a second bore with a mating feature for cooperating with a second actuator shaft of a surgical instrument to move the second translation member in the longitudinal direction.

In embodiments, the upper plate comprises first and second proximal ramps and first and second distal ramps extending towards the lower plate each in the proximal direction. The first translation member comprises a movable wedge with first and second upper proximal angled surfaces for cooperating with the first and second proximal ramps of the upper plate and first and second distal angled surfaces for cooperating with the first and second distal ramps of the upper plate.

In embodiments, the second translation member comprises a second movable wedge with at least one angled surface and the first translation member includes at least one angle surface or ramp positioned to cooperate with the second movable wedge of the second translation member. The second movable wedge and the ramp of the first translation member are positioned on the first side of the plates such that longitudinal movement of the second translation member causes the first side of the upper plate to move towards or away from the first side of the lower plate.

In embodiments, the first and second bores of the translation members each include a mating feature for securing the translation members to the actuator shafts. In one such embodiment, the mating feature comprises at least one recess formed within the bores for receiving at least one projection on the first and second actuator shafts. Rotation of the actuator shafts within the bores causes the projection(s) to rotate into the recess(es), thereby securing the actuator shafts to the translation members.

In embodiments, the device further comprises a mechanism for providing discrete steps that correspond to incremental changes in angle and/or height of the plates. In one such embodiment, the first and second ratchet shafts extend into a distal portion of the first and second bores of the translation members. The ratchet shafts comprise external projections extending along the shafts, such as teeth, threads or the like. The distal portion of the first and second bores each comprise two or more elongate members (e.g., fingers) separated from each other and configured to mate with the external projections on the ratchet shafts. This cooperation between the ratchet shafts and the fingers inhibits reverse (i.e., distal) movement of the translation members and creates discrete “steps” in the proximal movement of the translation members relative to the upper and lower plates. These steps correspond to height and angle adjustments or increments of the endplates.

In certain embodiments, the elongate members generally press inward against the projections of the ratchet shafts to prevent reverse movement of the translation members in the distal direction. The elongate members of the first and second bores may be configured to mate with a tapered distal end of the first and second actuator shafts of the instrument such that distal movement of the first and second actuator shafts causes the tapered distal end to engage the projections and urge the fingers laterally outward. This causes the internal ratchets to disengage from the projections such that distal or reverse movement of the translation members is possible.

In embodiments, the device may be part of a system that includes a third plate coupled to, or formed integrally with, a proximal surface of at least one of the upper and lower plates. This third plate includes one or more through-holes for receiving fasteners therethrough. The fasteners may include any suitable device, such as cannulated or uncannulated screws, bolts, nails, anchors or the like. The through-holes are oriented such that the fasteners can be advanced into the first and second vertebral bodies in the direction of, but spaced from, the outer surfaces of the upper and lower plates to secure the device to the vertebral bodies.

In embodiments, the system further includes a locking element coupled to the through-holes of the fixation plate. The locking element is movable from an unlocked position, wherein the fasteners may be passed through the through-holes, and a locked position, wherein the fasteners are secured within the through-holes. In an exemplary embodiment, the locking element comprises a rotatable element and a spring. Rotation of the rotatable element moves the spring into engagement with the through-hole and into the locking position. This prevents the fasteners from backing out or otherwise loosening from the vertebral bodies.

In another aspect, a spinal fusion system comprises an adjustable spinal fusion device having an upper endplate with an outer surface for placement against a first vertebral body and a lower endplate with an outer surface for placement against a second vertebral body. The device includes a first translation member configured to move longitudinally relative to the upper and lower plates to adjust a distance between the upper and lower plates and a second translation member configured to move longitudinally relative to the upper and lower plates to adjust an angle between the upper and lower plates. The system further comprises an instrument having a proximal handle, an elongate shaft and an actuator within the elongate shaft coupled to the proximal handle for moving the first and second translation members longitudinally relative to the upper and lower endplates.

In embodiments, the actuator comprises a first actuator shaft coupled to the first translation member and a second actuator shaft coupled to the second translation member. In one such embodiment, the actuators are laterally spaced from each other in the instrument and coupled to a user interface on the handle. In embodiments, the user interface comprises first and second rotatable knobs coupled to the first and second actuator shafts, respectively, such that rotation of the knobs causes longitudinal movement of the actuator shafts and the translation members.

In embodiments, the upper and lower plates each have proximal and distal ends and first and second sides extending from the proximal end to the distal end. The second translation member is configured to adjust the angle between the first and second sides to allow the upper plate to rotate relative to the lower plate around an axis substantially parallel to the longitudinal axis of the device.

In embodiments, the device comprises a hinge on one of the first and second sides for rotatably coupling the upper plate to the lower plate. The hinge may include a rod extending substantially longitudinally along one side of the plates. The upper plate may comprise one coupling elements, such as knuckles or the like, rotatably coupled around the rod for allowing rotation of the upper plate relative to the lower plate.

In embodiments, longitudinal movement of the first translation member causes longitudinal movement of the second translation member to adjust the distance between the upper and lower plates and longitudinal movement of the second translation member relative to the first translation member adjusts the angle between the upper and lower plates.

In embodiments, the first translation member comprises a first bore with a mating feature for cooperating with a first actuator shaft of a surgical instrument to move the first translation member in the longitudinal direction. The second translation member comprises a second bore with a mating feature for cooperating with a second actuator shaft of a surgical instrument to move the second translation member in the longitudinal direction.

In embodiments, the upper plate comprises first and second proximal ramps and first and second distal ramps extending towards the lower plate each in the proximal direction. The first translation member comprises a movable wedge with first and second upper proximal angled surfaces for cooperating with the first and second proximal ramps of the upper plate and first and second distal angled surfaces for cooperating with the first and second distal ramps of the upper plate.

In embodiments, the second translation member comprises a second movable wedge with at least one angled surface and the first translation member includes at least one angle surface or ramp positioned to cooperate with the second movable wedge of the second translation member. The second movable wedge and the ramp of the first translation member are positioned on the first side of the plates such that longitudinal movement of the second translation causes the first side of the upper plate to move towards or away from the first side of the lower plate.

In embodiments, the first and second bores of the translation members each include a mating feature for securing the translation members to the actuator shafts. In one such embodiment, the mating feature comprises at least one recess formed within the bores for receiving at least projection on the first and second actuator shafts. Rotation of the actuator shafts within the bores causes the projection(s) to rotate into the recess(es), thereby securing the actuator shafts to the translation members.

In embodiments, the device further comprises a mechanism for providing discrete steps that correspond to incremental changes in angle and/or height of the plates. In one such embodiment, the first and second ratchet shafts extending into a distal portion of the first and second bores of the translation members. The ratchet shafts comprise external projections extending along the shafts, such as teeth, threads or the like. The distal portion of the first and second bores each comprise two or more elongate members (e.g., fingers) separated from each other and configured to mate with the external projections on the ratchet shafts. This cooperation between the ratchet shafts and the fingers inhibits reverse (i.e., distal) movement of the translation members and creates discrete “steps” in the proximal movement of the translation members relative to the upper and lower plates. These steps correspond to height and angle adjustments or increments of the endplates.

In certain embodiments, the elongate members generally press inward against the projections of the ratchet shafts to prevent reverse movement of the translation members in the distal direction. The elongate members of the first and second bores may be configured to mate with a tapered distal end of the first and second actuator shafts of the instrument such that distal movement of the first and second actuator shafts causes the tapered distal end to engage the projections and urge the fingers laterally outward. This causes the internal ratchets to disengage from the projections such that distal or reverse movement of the translation members is possible.

In embodiments, the device may be part of a system that includes a third plate coupled to, or formed integrally with, a proximal surface of at least one of the upper and lower plates. This third plate includes one or more through-holes for receiving fasteners therethrough. The fasteners may include any suitable device, such as cannulated or uncannulated screws, bolts, nails, anchors or the like. The through-holes are oriented such that the fasteners can be advanced into the first and second vertebral bodies in the direction of, but spaced from, the outer surfaces of the upper and lower plates to secure the device to the vertebral bodies.

In embodiments, the system further includes a locking element coupled to the through-holes of the fixation plate. The locking element is movable from an unlocked position, wherein the fasteners may be passed through the through-holes, and a locked position, wherein the fasteners are secured within the through-holes. In an exemplary embodiment, the locking element comprises a rotatable element and a spring. Rotation of the rotatable element moves the spring into engagement with the through-hole and into the locking position. This prevents the fasteners from backing out or otherwise loosening from the vertebral bodies.

In another aspect, an adjustable spinal fusion device comprises an upper plate having an outer surface for placement against a first vertebral body, proximal and distal ends and first and second sides extending from the proximal end to the distal end and a lower plate having an outer surface for placement against a second vertebral body, proximal and distal ends and first and second sides extending from the proximal end to the distal end. The device further comprises a hinge on the second sides of the upper and lower plates for rotatably coupling the upper plate to the lower plate and a translation member configured to move longitudinally relative to the upper and lower plates to rotate the upper plate relative to the lower plate and adjust a distance between the first side of the upper plate and the first side of the lower plate.

The hinge may comprise a rod extending in a longitudinal direction along the second sides of the upper and lower plates. The upper plate may comprise one or knuckles or other coupling elements rotatably coupled to the rod. The one or more knuckles may have a hollow interior with a height larger than an outer diameter of the rod to allow the second side of the upper plate to move relative to the second side of the lower endplate.

The device may include a second translation member configured to move longitudinally relative to the upper and lower plates to adjust a height between the upper and lower plates. The second translation member may comprise a knuckle for receiving the rod and at least one angled surface positioned to cooperate with a ramp of the first translation member. The ramp and the angled surface may be positioned on the first side of the plates such that longitudinal movement of the first translation causes the first side of the upper plate to move towards and away from the first side of the lower plate.

It is to be understood that both the foregoing general description and the following detailed description are exemplary and explanatory only and are not restrictive of the disclosure. Additional features of the disclosure will be set forth in part in the description which follows or may be learned by practice of the disclosure.

### BRIEF DESCRIPTION OF THE DRAWINGS

The accompanying drawings, which are incorporated in and constitute a part of this specification, illustrate several embodiments of the disclosure and together with the description, serve to explain the principles of the disclosure.

FIG. 1 is a perspective view of an implantable spinal system;

FIG. 2A is a proximal perspective view adjustable spinal device of the system of FIG. 1;

FIG. 2B is a distal perspective view of the spinal device;

FIG. 3 is a top view of the device;

FIG. 4 is a partially exploded view of the device;

FIG. 5 is a fully exploded view of the device;

FIG. 6A is a top view of an upper endplate of the spinal device;

FIG. 6B is a bottom view of the upper endplate;

FIG. 7 is a perspective view of a bottom portion of a height translation member of the spinal device;

FIG. 8 is a perspective view of an angle translation member of the spinal device;

FIG. 9 is a perspective view of a top portion of the height translation member;

FIG. 10 is a partial cross-sectional view of the spinal device;

FIG. 11 is a cross-sectional view of one portion of the height translation member;

FIG. 12 is an internal view of a distal portion of the spinal device;

FIG. 13 illustrates a ratchet shaft and one portion of the angle translation member;

FIG. 14 is a partial cross-sectional view of the spinal device;

FIG. 15 illustrates internal ratchets on the height and angle translation members;

FIG. 16 is another partial cross-sectional view of the spinal device;

FIG. 17 illustrates the spinal device with an angle between the upper and lower endplates;

FIG. 18 is a partially transparent bottom view of the spinal device;

FIG. 19A is a side view of the spinal device in a configuration for insertion between adjacent vertebral bodies;

FIG. 19B is a side view of the spinal device with an adjustment in height;

FIG. 19C is a side view of the spinal device with an adjustment in height and angle;

FIG. 20 is a perspective view of an instrument for inserting the spinal device between adjacent vertebral bodies;

FIG. 21 is a partially exploded view of the spinal device and a distal portion of the surgical instrument;

FIG. 22 is a cut-a-way view of a portion of the spinal device and the distal portion of the instrument;

FIG. 23 is a partial exploded view illustrating components of the spinal device and the instrument;

FIG. 24 is a partially exploded view of a proximal handle of the instrument;

FIG. 25 illustrates a rotatable knob of the handle;

FIG. 26 illustrates an indicia of the rotatable knob;

FIG. 27 illustrates a proximal side of the spinal system of FIG. 1;

FIG. 28 illustrates a distal side of the spinal system;

FIG. 29A illustrates first and second fasteners prior to insertion into through-holes in a proximal plate of the spinal system;

FIG. 29B illustrates the fasteners inserted into the through-holes in the proximal plate of the system;

FIG. 30 is a close up view of one fastener and its through-hole;

FIG. 31 illustrates a locking element for the fastener in an unlocked position;

FIG. 32 illustrates the locking element in the locked position.

FIG. 33 illustrates another embodiment of an instrument for inserting the spinal device between adjacent vertebral bodies;

FIG. 34 is a partially exploded view of a distal end of the instrument of FIG. 33;

FIG. 35 is an enlarged view of a distal end of two actuator shafts of the instrument of FIG. 33;

FIG. 36 is an exploded view of a proximal end of the instrument of FIG. 33;

FIG. 37 illustrates a trial implant and an instrument for inserting the trial implant between adjacent vertebral bodies;

FIG. 38 is an exploded view of a distal portion of the trial implant and instrument of FIG. 37; and

FIG. 39 is an exploded view of a proximal portion of the trial implant and instrument of FIG. 38.

### DESCRIPTION OF THE EMBODIMENTS

This description and the accompanying drawings illustrate exemplary embodiments and should not be taken as limiting, with the claims defining the scope of the present disclosure, including equivalents. Various mechanical, compositional, structural, and operational changes may be made without departing from the scope of this description and the claims, including equivalents. In some instances, well-known structures and techniques have not been shown or described in detail so as not to obscure the disclosure. Like numbers in two or more figures represent the same or similar elements. Furthermore, elements and their associated aspects that are described in detail with reference to one embodiment may, whenever practical, be included in other embodiments in which they are not specifically shown or described. For example, if an element is described in detail with reference to one embodiment and is not described with reference to a second embodiment, the element may nevertheless be claimed as included in the second embodiment. Moreover, the depictions herein are for illustrative purposes only and do not necessarily reflect the actual shape, size, or dimensions of the system or illustrated components.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

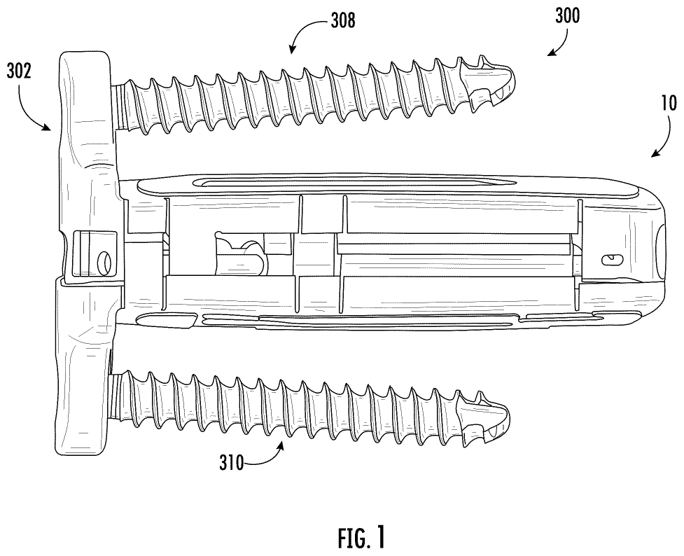

Referring now to FIG. 1, a spinal system 300 according to the present disclosure is configured for placement between two adjacent vertebral bodies. Spinal system 300 comprises a fixation plate 302 coupled to a spinal implant device 10. Fixation plate 302 includes first and second through-holes 304, 306 (see 28) for receiving first and second fixation devices, or fasteners, 308, 310 that extend on either side of device 10 for fixation to the two vertebral bodies, as discussed in more detail below. In some embodiments, implant 10 is particularly useful for advancement via a direct lateral approach through the psoas muscle (lateral lumbar interbody fusion or LLIF), although it will be recognized that the systems and implants disclosed herein may be employed in a variety of different surgical approaches, such as anterior lumbar interbody fusion (ALIF), posterior lumbar interbody fusion (PLIF) and/or (transforaminal lumbar interbody fusion or TLIF).

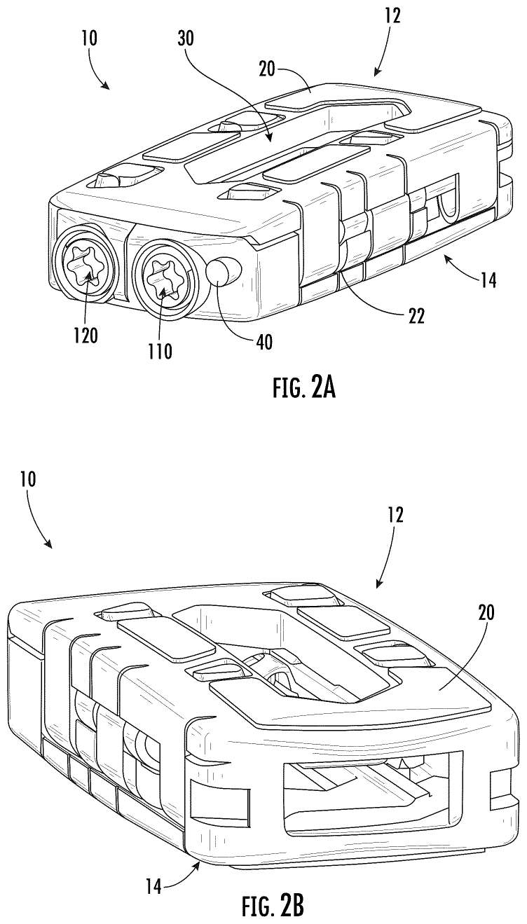

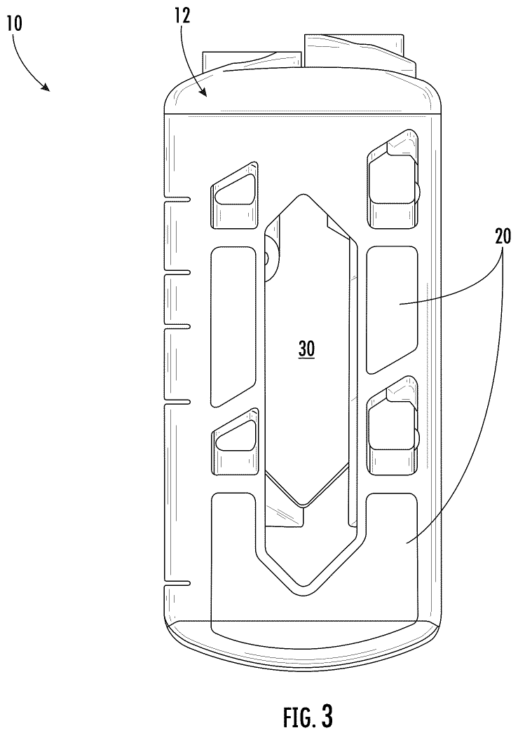

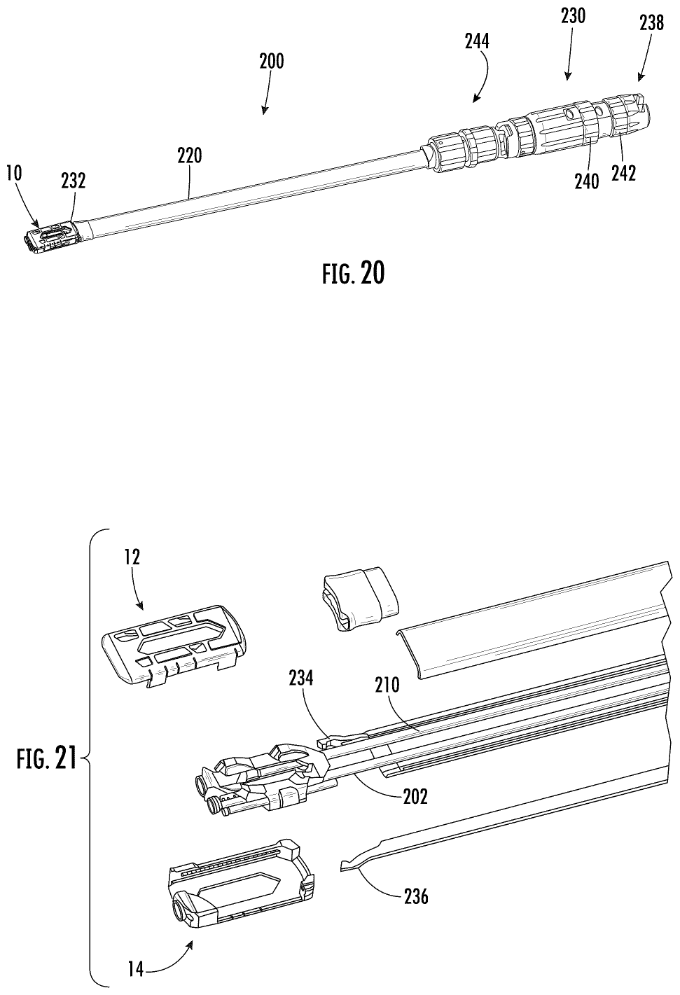

Referring now to FIGS. 2-9, spinal implant device 10 includes upper and lower endplates 12, 14, an angle translation member 16 and a height translation member 18. The height and angle translation members 16, 18 are configured to be translated in the longitudinal direction relative to the endplates 12, 14 by first and second shafts 202, 210 of an insertion instrument 200 (see FIGS. 20 and 21). Movement of the translation members 16, 18 in substantially the longitudinal direction changes the height and angle of endplates 12, 14 as discussed in more detail below.

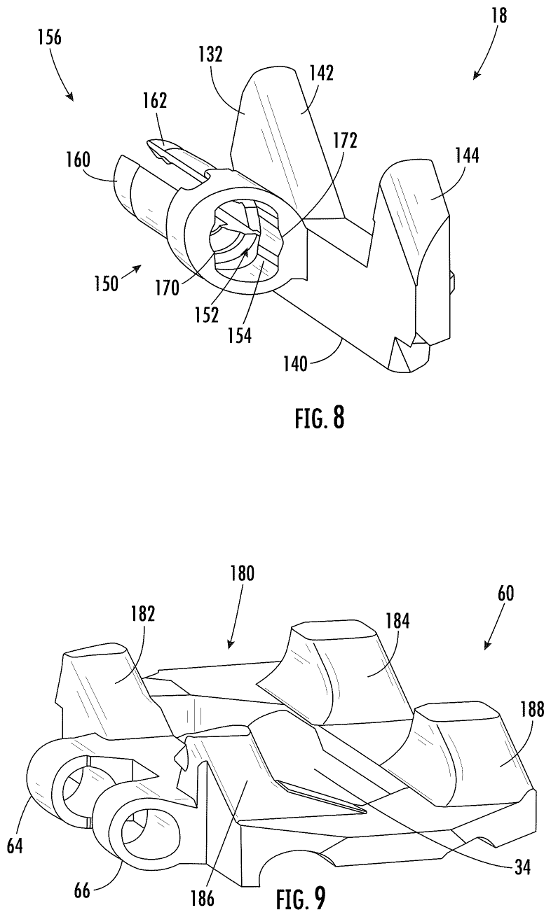

Upper and lower endplates 12, 14 each include an outer surface 20, 22, respectively, for contacting the surface of a vertebral body. Outer surfaces 20, 22 are preferably roughened with a surface treatment that facilitates attachment to the vertebral body. The surface treatment preferably creates a diamond structure (e.g., diamond 20-1.5), although other patterns are contemplated. Upper and lower endplates 12, 14 each include central openings 30, 32 that extend through the entire endplates 12, 14 and, in one embodiment, are substantially aligned with each other. Similarly, height translation member 18, includes a central opening or bore 34 that, in one embodiment, may be substantially aligned with endplate openings 30, 32 (see FIG. 9). These openings create space for the addition of bone graft or other substances into the implant, as well as to allow for bony ingrowth through implant 10.

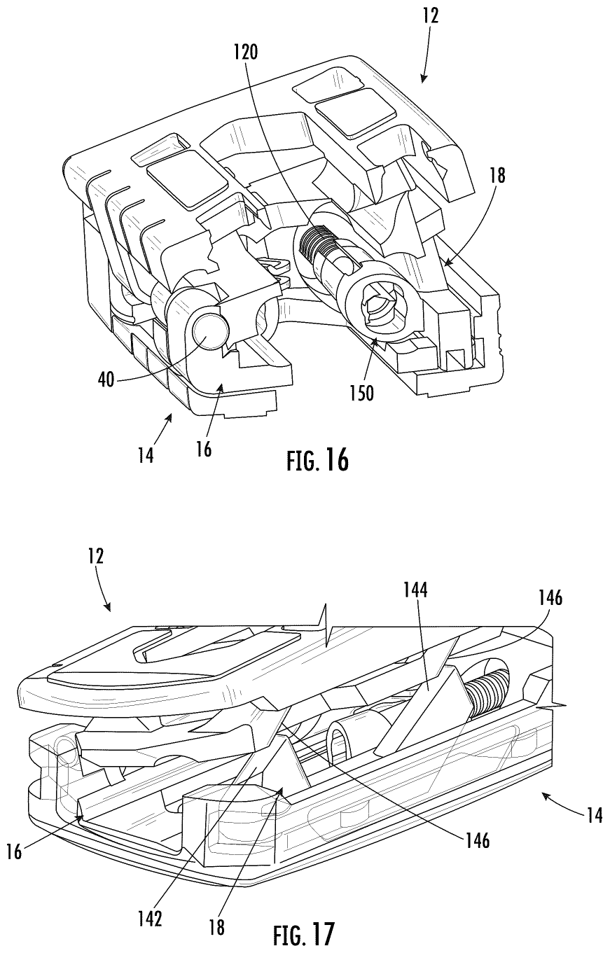

Upper and lower endplates 12, 14 each comprises proximal and distal ends 21, 23 and first and second sides 25, 27 extending between proximal and distal ends 21, 23. Endplates 12, 14 are connected to each other via a hinge located along first side 25 of the endplates, which allows upper endplate 12 to rotate relative to lower endplate 14 to increase or decrease the distance between second sides 27 of endplates 12, 14, thereby changing the overall angle of device 10 once it is positioned between the vertebral bodies in the patient (see, for example, FIG. 16).

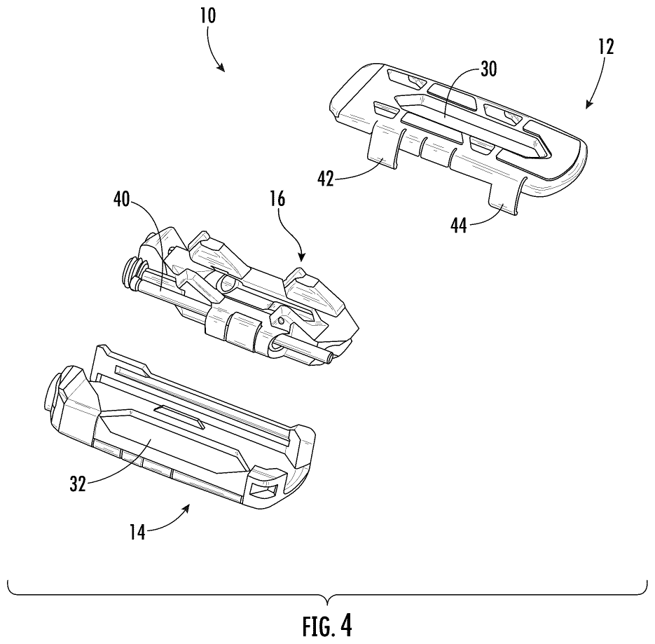

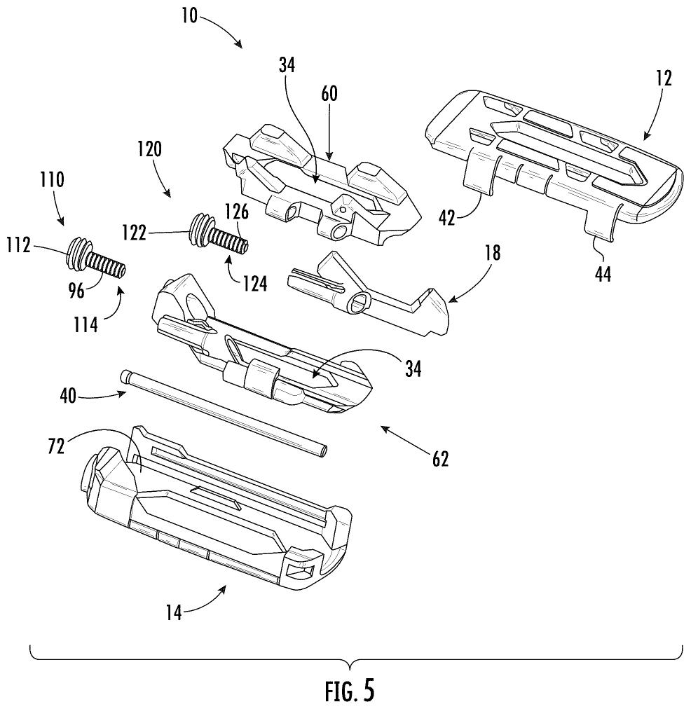

Referring now to FIGS. 4 and 5, in certain embodiments the hinge comprises a rod 40 that extends in the longitudinal direction alongside 25 of lower endplate 14 and through first and second coupling elements 42, 44 in upper endplate 12 (see FIG. 4). Coupler elements 42, 44 may comprise any suitable component that allows for rotation around rod 40, such as knuckles or the like. Knuckles 42, 44 are preferably U-shaped substantially hollow components extending downward from side 25 of upper endplate 12. Knuckles 42, 44 allow rotation of upper endplate 12 relative to lower endplate 14 around the longitudinal axis defined by rod 40. In addition, the hollow interiors of knuckles 42, 44 preferably have a larger height than the outer diameter of rod 40 to allow the upper endplate 12 to move vertically relative to the lower endplate 14 while still retaining rod 40 within knuckles 42, 44, as discussed below.

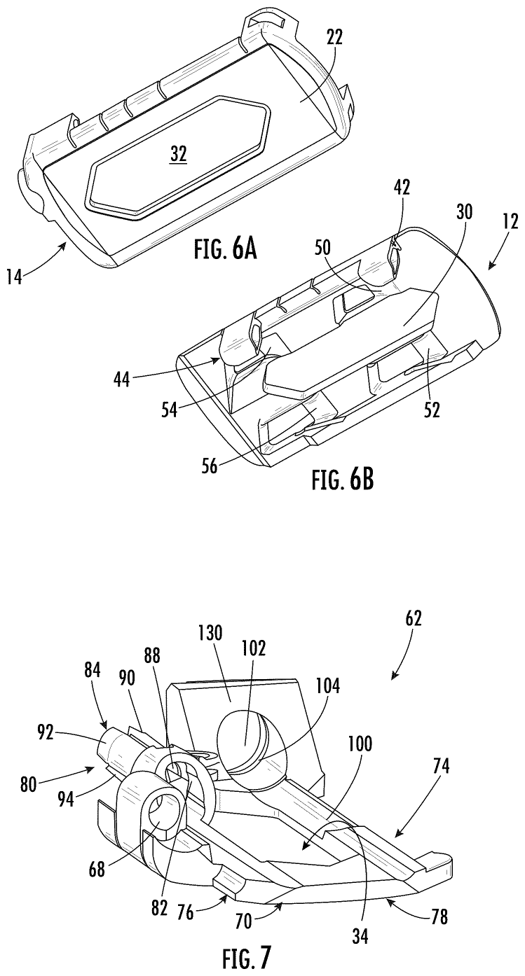

As shown in FIG. 6B, upper endplate 12 includes: (1) first and second distal sloped surfaces or ramps 50, 52 that are laterally spaced from each other near either side of the upper endplate 12 and extend towards the lower endplate 14 in the proximal direction; and (2) third and fourth proximal sloped surfaces or ramps 54, 56 that are laterally spaced from each other near either side of upper endplate 12 and also extend towards the lower endplate 14 in the proximal direction. These ramps interact with wedges on the height translation member 16 to provide height adjustment of the implant.

In an alternative embodiment, upper endplate 12 may include a single distal ramp and/or a single proximal ramp that extends laterally across a central portion of the endplate 12. Alternatively, endplate 12 may include more than two distal or proximal ramps.

As shown in FIGS. 7 and 9, height translation member 16 includes an upper portion 60 and a lower portion 62 that may be integral with each other, coupled to each other or completely separate components from each other. Upper portion 60 includes first and second hinge cylinders 64, 66 on one of the sides of the upper portion 60 spaced longitudinally from each other. Hinge cylinders 64, 66 each have a hollow lumen configured to receive rod 40 of the hinge, which extends through the lumens of first and second cylinders 64, 66. These cylinders 64, 66 couple the upper portion 60 of the height translation member 16 to the lower endplate 14 and allow this portion to slide longitudinally relative to the endplates along the rod 40. The lower portion 62 also includes a hinge cylinder 68 on one side of lower portion 62 that is positioned between the first and second cylinders 64, 66 of upper portion 60. This cylinder 68 has a hollow lumen that receives rod 40 of the hinge, thereby coupling lower portion 60 to lower endplate 14 and allowing it to slide longitudinally along the rod 40 relative to the endplates.

Upper portion 60 of the height translation member 16 comprises a main body 180 with: (1) first and second distal wedges 182, 184 for engaging with distal ramps 50, 52 on the upper endplate 12 and (2) first and second proximal wedges 186, 188 for engaging with proximal ramps 54, 56 of the upper endplate. Longitudinal movement of translation member 16 causes the upper endplate to move towards and away from the lower endplate. In an alternative embodiment, distal translation member 16 may include a single distal ramp and/or a single proximal ramp that extends laterally across a central portion of the translation member 16. Alternatively, translation member 16 may include more than two distal or proximal ramps.

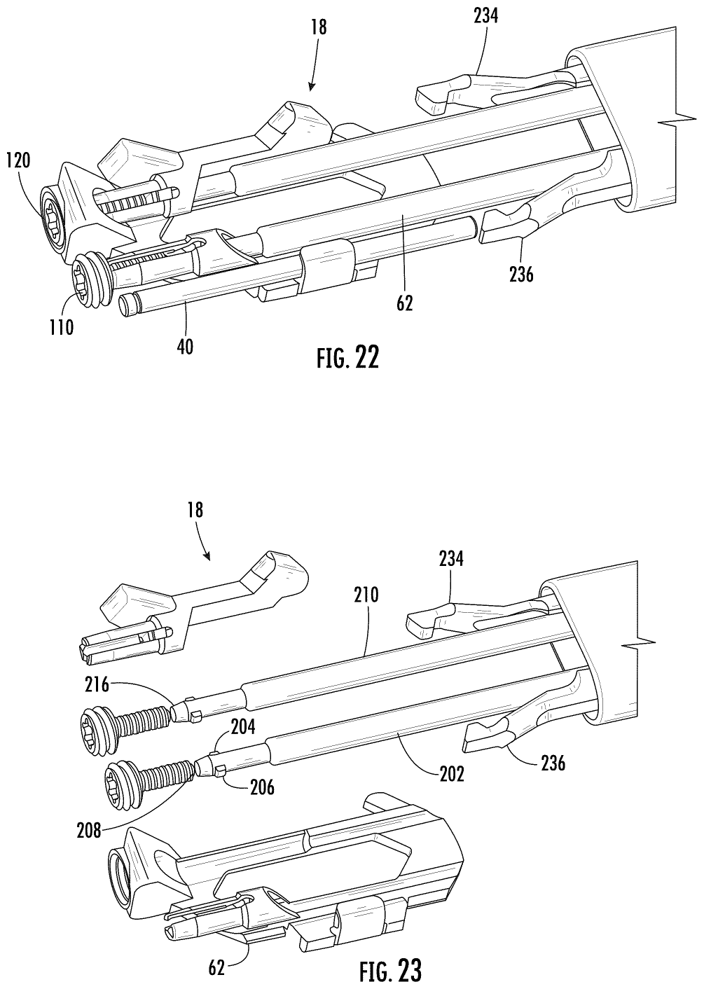

As shown in FIG. 7, lower portion 62 of height translation member 16 comprises a frame 70 generally positioned on, or adjacent to, an upper surface 72 of lower endplate 14 (see FIG. 5). Frame 70 comprises first and second lateral elements 74, 76 that extend longitudinally on either side of central opening 34 of lower portion 62. Lateral elements 74, 76 are coupled to each other by a proximal base portion 78. The first lateral element 76 comprises a bore 80 with a proximal opening 82 for receiving a first shaft actuator 202 on the insertion instrument 200 and a distal opening 84 for receiving a first ratchet shaft 110 having a threaded head or attachment end (see FIG. 22).

The proximal portion of bore 80 includes a mating feature for securing lower portion 62 of height translation member 16 to first shaft actuator shaft 202. In one embodiment, this mating feature comprises one or more recesses (not shown) formed within bore 80 and extending laterally inward from the central axis of the bore 80. These recesses are designed to accommodate one or more projections 204, 206 on the distal end of first instrument actuator shaft 202 (see FIG. 23). These projections 204, 206 can be rotated into the recesses to lock actuator shaft 202 to lower portion 62 of height translation member 16. Once this occurs, longitudinal translation of actuator shaft 202 causes longitudinal translation of lower portion 62 of height translation member 16.

First ratchet shaft 110 includes a threaded head 112 and a shaft 114 with a series of external projections, such as teeth 96, extending therefrom (see FIG. 5). Shaft 144 110 is received within bore 80 of lower portion 62 of translation member 18. The distal portion of bore 80 is split into three separate elongate members or fingers 90, 92, 94 that are coupled together at a proximal end and split apart from each other in the distal direction. The three fingers 90, 92, 94 each comprise internal ratchets (not shown) that mate with external teeth 96 on first ratchet shaft 110 (see FIG. 5) such that proximal movement of lower portion 62 of the height translation member 16 causes the internal ratchets to pass over each of the teeth 96 on ratchet shaft 110 to create discrete “steps” in the movement of the height translation member 16 relative to the endplates 12, 14. These steps correspond to height adjustments or increments of the endplates.

The three fingers 90, 92, 94 generally press inward against the threads or teeth 96 of ratchet shaft 110 to prevent reverse movement of height translation member 16 in the distal direction. To provide this reverse movement, each of the fingers 90, 92, 94 include internal projections 98 (see FIG. 11A) on their proximal ends designed to mate with a tapered distal end 208 of the instrument actuator shaft 202 (see FIG. 23). These internal projections 98 are designed such that distal movement of actuator shaft 202 causes tapered distal end 208 to engage projections 98 and urge the fingers laterally outward. As fingers 90, 92, 94 are urged laterally outward, the internal ratchets disengage from threads 96 of the ratchet shaft 110 such that distal movement of height translation member 16 is possible.

Referring again to FIG. 7, second lateral element 74 of lower portion 62 of the height translation member 16 has an upper surface 100 and a distal opening or bore 102 with internal threads 104. Angle translation member 18 is positioned on, or near, upper surface 100 of this lower portion 62. A second ratchet shaft 120 includes a threaded head 122 and a shaft 124 with a series of external teeth 126 extending therefrom (see FIG. 5). The threaded head 122 of the second ratchet shaft 120 is received within this bore 102.

Lower portion 62 of the height translation member 16 further comprises a distal wedge surface 130 positioned to contact and engage a distal surface 132 of the angle translation member 18 (see FIG. 8). This distal wedge surface 130 contacts angle translation member 18 such that, when height translation member 16 is moved in a proximal direction, it moves angle translation member 18 therewith.

As shown in FIG. 18, height translation member 16 further comprises runners 180 positioned at the outer surface of height translation member adjacent to the horizontal surface of lower endplate 14 and configured to lower the friction therebetween. Alternatively, or in addition runners 180 can be provided within lower endplate 14.

Referring now to FIG. 8, angle translation member 18 comprises an elongate body 140 extending in a longitudinal direction and positioned over the upper surface of one of lateral element 74 of lower portion 62 of height translation member 16. This elongate body 140 includes first and second wedges 142, 144 having a sloped surface that extends downwards in the proximal direction. These wedges 142, 144 are positioned to contact and engage ramps 146 on one side of upper portion 60 of height translation member 16. Ramps 146 extend downward from the lower surface of this upper portion 60 (see FIG. 17). Longitudinal movement of angle translation member 18 causes wedges 142, 144 to engage ramps 146 and move one side of height translation member 16 towards or away from lower endplate 14. Height translation member 16, in turn, causes one side of upper endplate 12 to move towards or away from lower endplate 14. Since the other side of upper endplate 12 remains fixed in place during this movement, the angle of upper endplate 12 relative to lower endplate 14 is thereby adjusted.

Angle translation member 18 further includes a cylindrical component 150 coupled to elongate body 140 and positioned closer to the longitudinal axis of implant 10. Cylindrical component 150 includes a central bore 152 with a proximal opening 154 for receiving a second shaft actuator 210 on the insertion instrument 200 and a distal opening 156 for receiving second threaded ratchet shaft 120. Bore 150 includes a mating feature, such as internal recesses 170, 172, for mating with projections 204, 206 of actuator shaft 210 (see FIG. 23) similar to the description above with respect to bore 80 of height translation member 16.

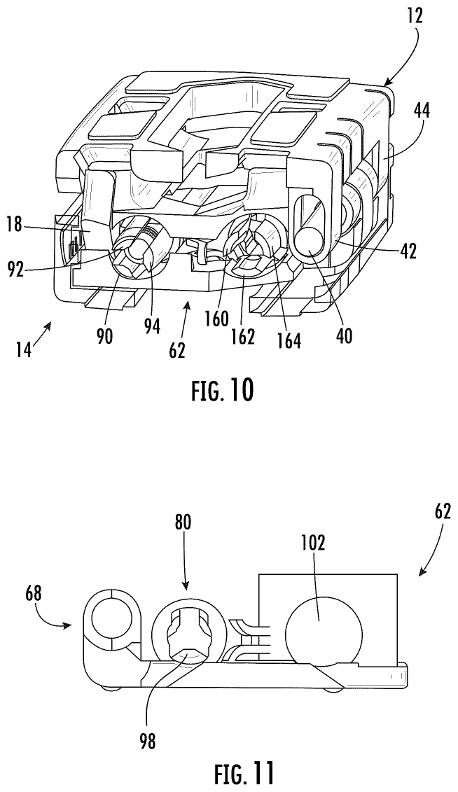

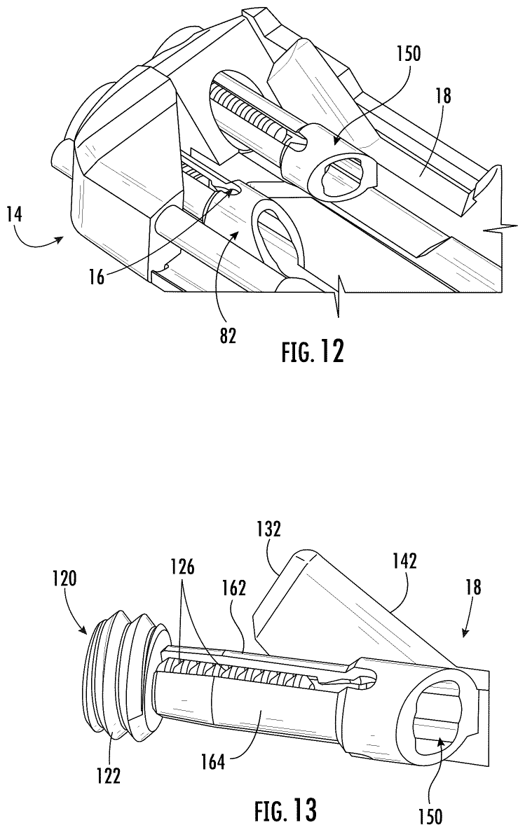

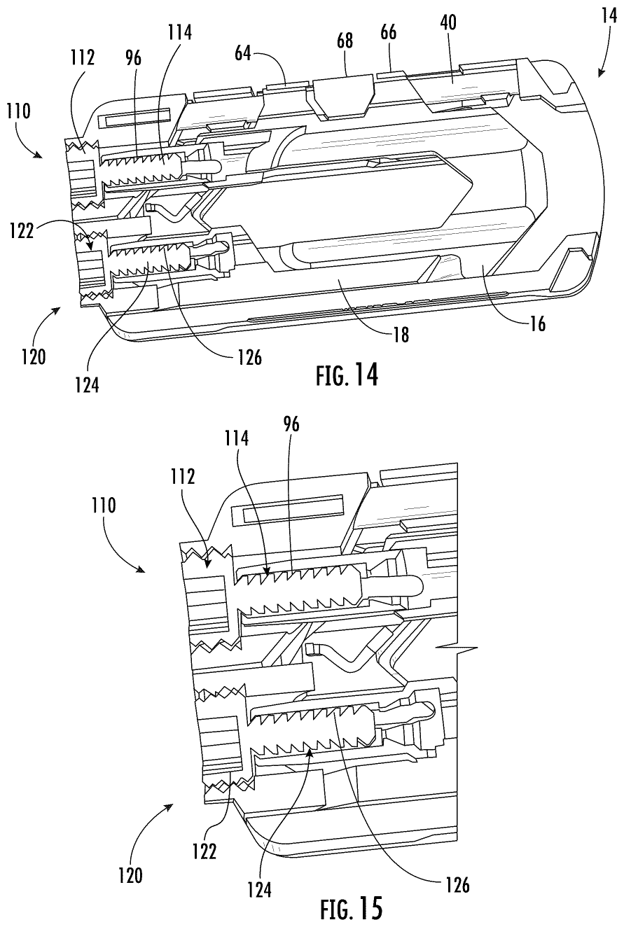

The distal portion of bore 152 may include separate and independently movable fingers or prongs. In one embodiment as shown in FIGS. 8 and 10, the bore 152 is split into three separate elongate members or fingers 160, 162, 164 that are coupled together at a proximal end and split apart from each other in the distal direction (see FIGS. 8 and 10). The three fingers 160, 162, 164 each comprise internal ratchets (not shown) that mate with second threaded ratchet shaft 120 such that proximal movement of angle translation member 18 causes the internal ratchets to pass over each of the threads 126 on ratchet shaft 120 to create discrete “steps” in the movement of the angle translation member 18 relative to the height translation member 16 and the endplates 12, 14 (see FIGS. 14 and 15). These steps correlate with angle adjustments of the endplates

The three fingers 160, 162, 164 generally press inward against the second ratchet shaft 120 to prevent reverse movement of the angle translation member 18 in the distal direction. To provide this reverse movement, each of the fingers 160, 162, 164 include internal projections 168 designed to mate with a tapered distal end 216 of the second instrument actuator shaft 210. These internal projections 168 (see FIG. 11C) are designed such that distal movement of the second actuator shaft 210 causes the tapered distal end 216 to engage the projections 168 and urge the fingers 160, 162, 164 laterally outward. As the fingers are urged laterally outward, the internal ratchets 166 disengage from the teeth 126 of the ratchet shaft 120 such that distal movement of the angle translation member 18 is possible.

It is understood, of course, that the bore 152 may be divided into any number of separate and independent fingers or prongs, each of the fingers being configured as the ones shown and described above and in FIGS. 8 and 10. Accordingly, the number of fingers provided may vary without departing from their desired function. For example, in another embodiment, the bore 152 is provided with four fingers configured as two pairs of fingers on opposite sides of bore 152. In this embodiment, two of the fingers on one side of the bore are engaged by the second threaded shaft 120 while the opposite two fingers are not engaged by shaft 120. After a half-step of the ratchet shaft 120, the pair of fingers alternate such that that the opposite two fingers are engaged while the first pair of fingers are not engaged. This embodiment reduces the size or distance between each clicker step in the implant. In still other embodiments, the number of fingers may range from two to eight or more, depending on size limitations and desired travel range for the clicker step.

Referring now to FIGS. 20 and 21, insertion instrument 200 comprises an elongated shaft 220 with a proximal handle 230 and a distal gripping element 232 for removable coupling to the implant. Distal gripping element 232 includes first and second gripping arms 234, 236 for coupling to lower endplate 14 of the implant (a bayonet style connection). Distal gripping arms 234, 236 are coupled to an actuator 238 on proximal handle 230 to move arms 234, 236 in a substantially lateral direction relative to the longitudinal axis of shaft 220. Arms 234, 236 can be moved together to hold lower endplate 14 and moved apart to release the endplate.

Insertion instrument 200 further includes first and second actuator shafts 202, 210 extending from the handle 230 to the bores 80, 152 within the height and angle translation members 16, 18, respectively. First and second shafts 202, 210 are positioned on opposite lateral portions of the instrument and are both attached to rotatable knobs 240, 242 on the proximal handle for longitudinally translating the shafts. As shown in FIG. 23, each shaft 202, 210 includes a distal tapered end 208, 216 and at least two projections 204, 206 extending laterally outward from the shaft proximal of the distal tapered end. The two projections 204, 206 are designed to translate through a proximal opening of the bores 80, 152 on the height and angle translation members 16, 18. Rotation of the shafts 202, 210 causes the two projections 204, 206 to slide into the two recesses within these bores, thereby locking the shafts to the height and angle translation members. Rotation of the knobs 240, 242 on the handle then causes the shafts and the translation members to move longitudinally.

The distal tapered ends 208, 216 of the instrument shafts 202, 210 are designed to engage the three internal projections within the bores of the angle and height translation members. Proximal movement of the instrument shafts causes the distal tapered end to engage these projections and urge the three elongate distal members of the bores laterally outward to allow reverse or distal movement of the angle and translation members.

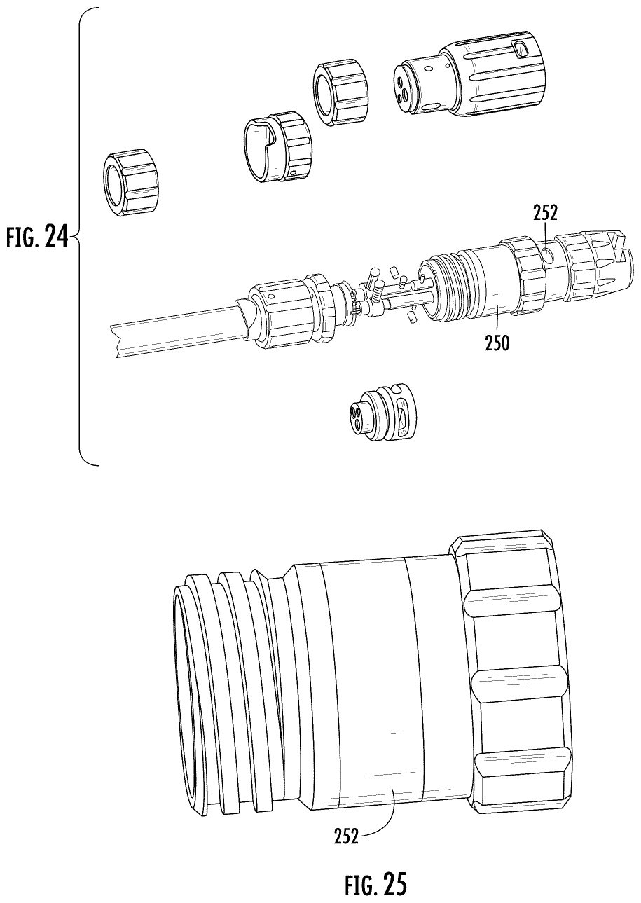

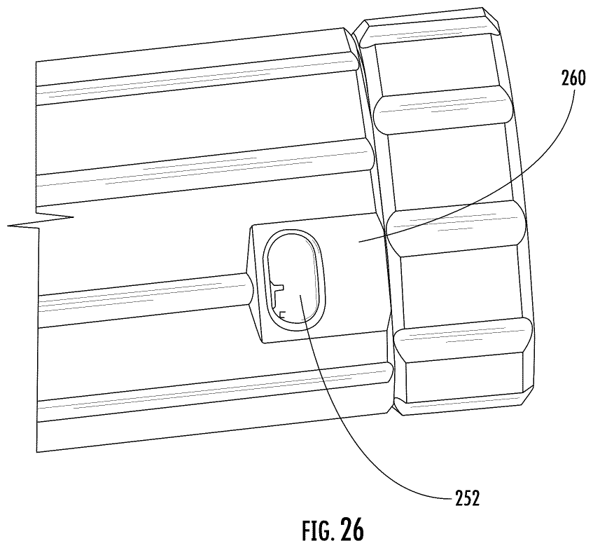

As shown in FIGS. 24-26, the proximal handle 230 comprises position indicators 250, 252 configured to indicate the position of each actuator shaft 202, 210, respectively. The position indicators 250, 252 may be formed in a recess 260 of handle 230.

The handle further includes a third rotatable knob 244 coupled to the shaft for rotating the shaft and the endplate therewith relative to the handle. This allows for rotation of the endplate without rotating the handle to facilitate ease of use during implantation.

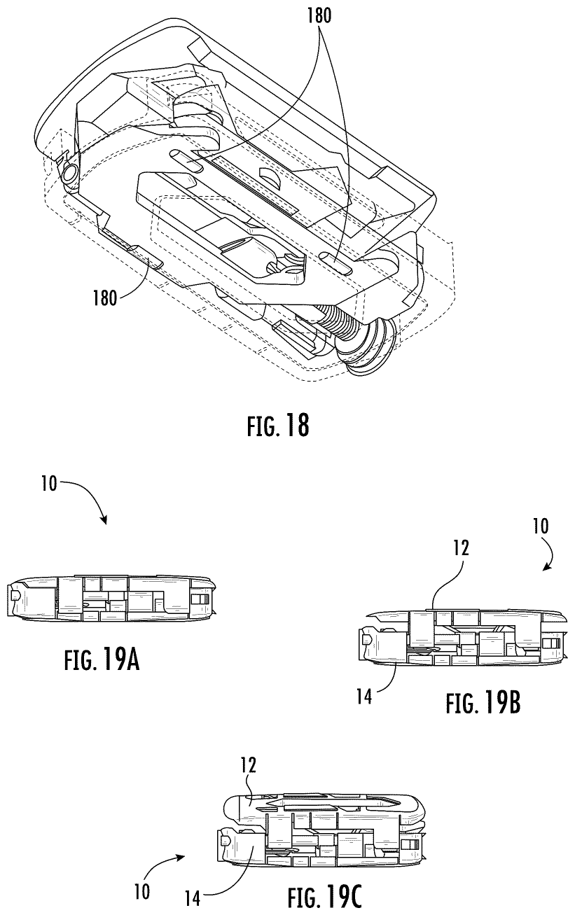

In use, the implant may be advanced into an intervertebral space in a collapsed configuration (see the resting state shown in FIG. 19A). To increase the height of the implant, the endplates are moved away from each other in a substantially parallel direction. To that end, the first and second instrument shafts are advanced distally through the bores of the angle and height translation members and rotated 90 degrees to lock the shafts to the translation members. The rotatable knob (green) on the handle is rotated to thereby withdraw its associated shaft proximally. This translates both the height and angle translation members in the proximal direction. As the height translation member moves in the proximal direction, its four upper wedges engage with the upper endplate ramps such that the endplates move apart from each other in a substantially parallel direction (see expanded state shown in FIG. 19B).

To adjust the angle of the endplates, the rotatable knob (red) on the handle is rotated, thereby withdrawing its associated shaft proximally. The angle translation member is translated in a proximal direction relative to both the endplates and the height translation member. The wedges on the angle translation member engage the ramps on the one side of the height translation member to move this side upwards away from the lower endplate, thereby adjusting the angle of the upper endplate relative to the lower endplate (see angularly adjusted state shown in FIG. 19C).

The process of height and angle adjustment is reversible. The height and angle may be adjusted independently of each other. For example, the above process can be reversed such that the shaft of the angle translation member is first rotated to adjust angle, and then the shaft of the height translation member is rotated to adjust height.

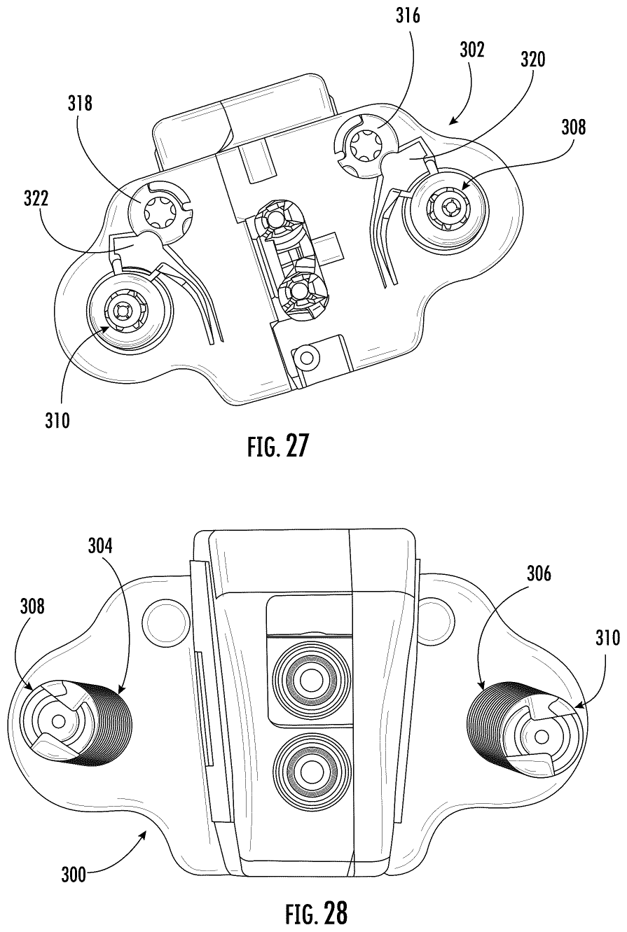

Referring now to FIGS. 27-32, plate 302 is coupled to, or formed integrally with, the proximal end of device 10 and includes through-holes 304, 306 for receiving fasteners 308, 310. Fasteners 308, 310 may comprise any suitable fastening device, such as cannulated or uncannulated screws, bolts, nails or the like. In one embodiment, fasteners 308, 310 comprise screws having a head, a threaded shaft and a distal tip for advancing the screws into adjacent vertebral bodies to secure device 10 between the vertebral bodies. Through-holes 304, 306 may be oriented such that fasteners 308, 310 extend substantially parallel to the outer surfaces of endplates 12, 14, or they may be oriented such that fasteners 308, 310 extend at an acute angle to endplates 12, 14 (as shown in FIG. 1).

As shown in FIG. 27, fixation plate 302 including a locking or anti-backout mechanism for securing fasteners 308, 310 to through-holes 304, 306. The locking mechanism includes locking screws 316, 318 and leaf springs 320, 322 positioned on a proximal surface of plate 302. Locking screws 316, 318 each include a mating feature for cooperating with an external instrument (not shown) to rotate screws 316, 318. Locking screws 316, 318 are coupled to leaf springs 320, 322 such that rotation of screws 316, 318 moves leaf springs 320, 322 towards through-holes 304, 306. Leaf springs 320, 322 are naturally biased away from through-holes 304, 306.

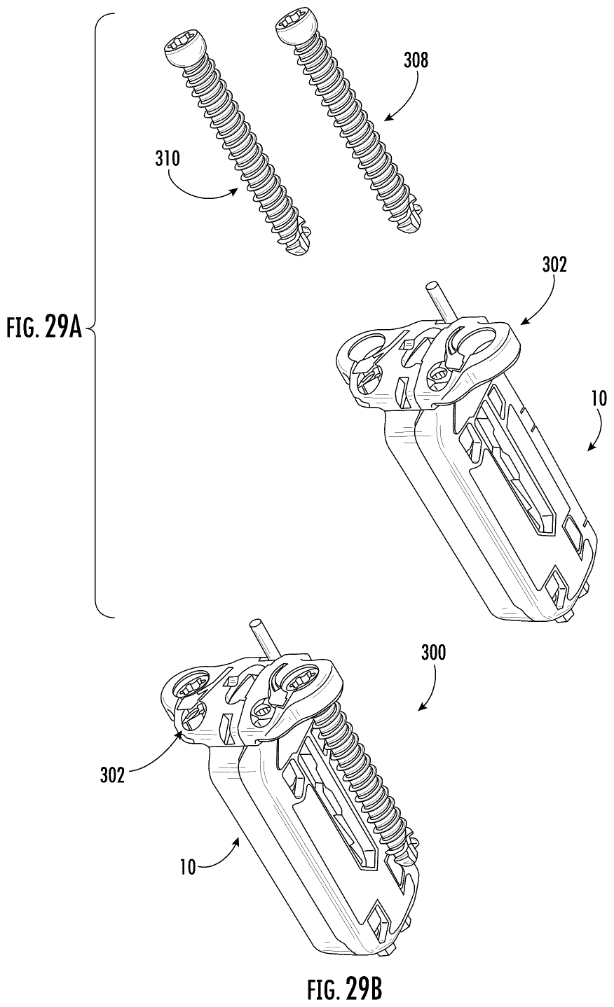

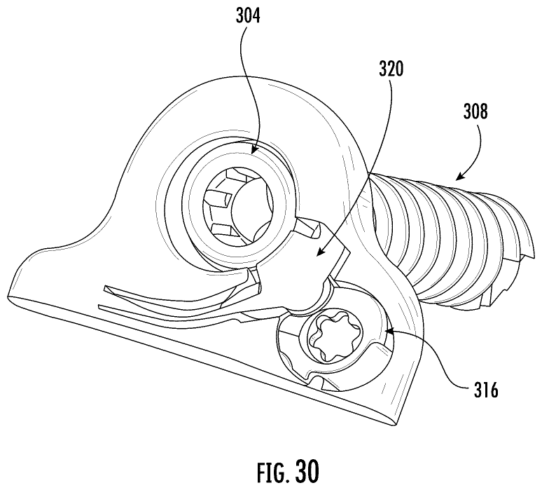

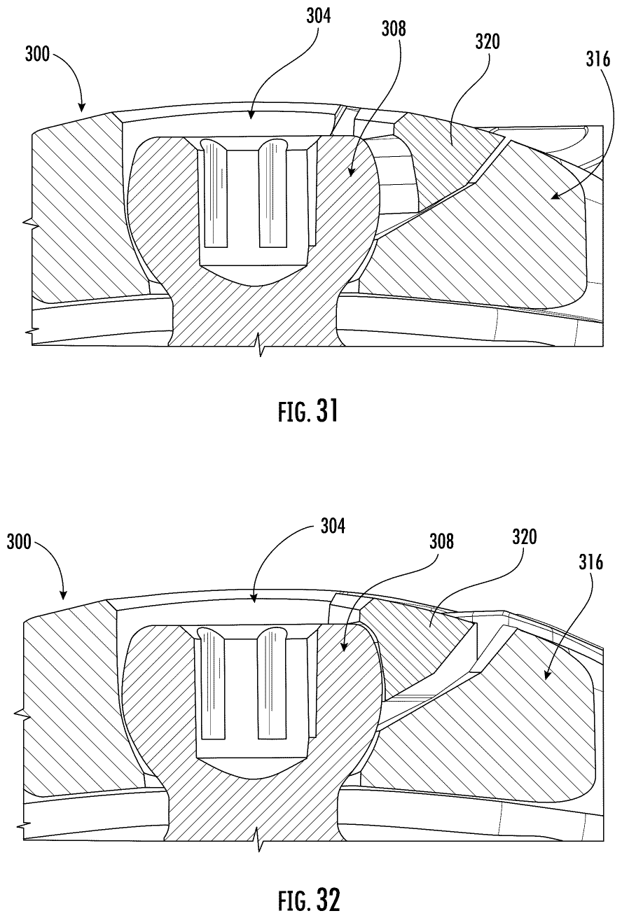

In use, locking screws 316, 318 start in the unlocked position (FIG. 31) such that leaf springs 320, 322 are spaced from through-holes 304, 306. Fasteners 318, 310 are advanced into the vertebral bodies to secure system in place (see FIGS. 29A and 29B). Once the fasteners have been fully advanced into the through-holes such that the heads of the fasteners rest within the proximal recesses 330 of plate 302, an instrument is used to rotate locking screws 316, 318 such that leaf springs 320, 322 move over the heads of the fasteners into the locked position (See FIG. 32). This prevents the fasteners from backing out or otherwise loosening from through-holes 304, 306.

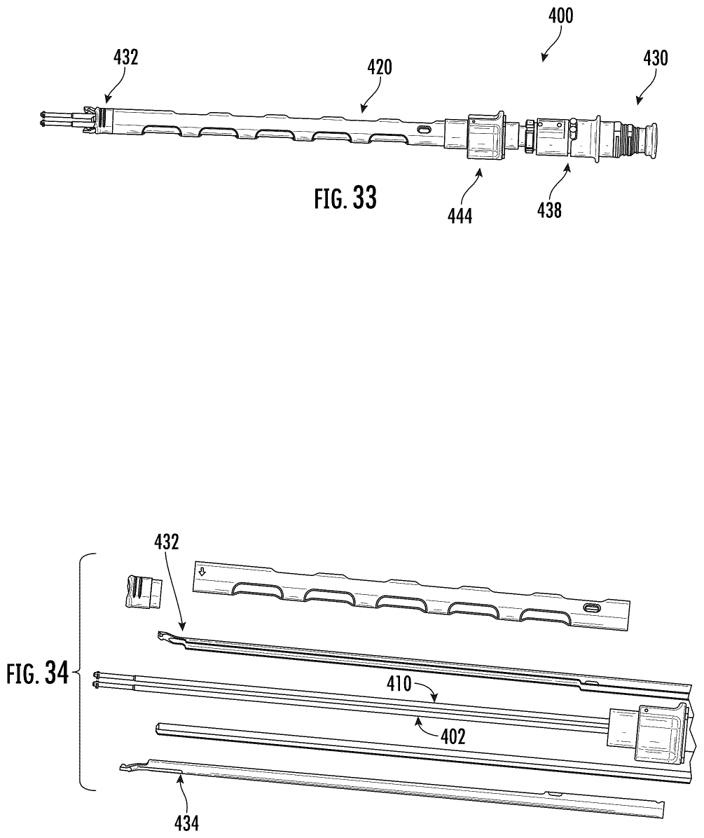

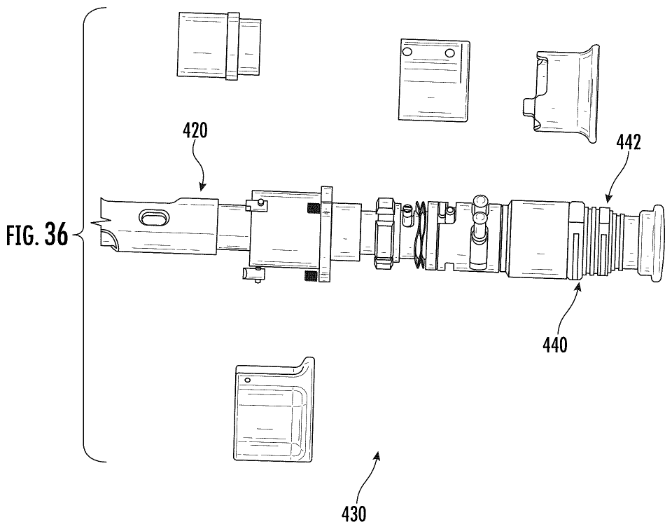

Referring now to FIGS. 33-36, another embodiment of an instrument 400 for inserting any of the implantable spinal devices described above between adjacent vertebral bodies will now be described. Similar to the previous embodiment, instrument 400 comprises an elongated shaft 420 with a proximal handle 430 and a distal gripping element 432 for removable coupling to the implant. Distal gripping element 432 includes first and second gripping arms 434, 436 for coupling to the lower and/or upper endplates of the implant (a bayonet style connection). Distal gripping arms 434, 436 are coupled to an actuator 438 on proximal handle 430 to move arms 434, 236 in a substantially lateral direction relative to the longitudinal axis of shaft 420. Arms 434, 436 can be moved together to hold the endplates and moved apart to release the endplates.

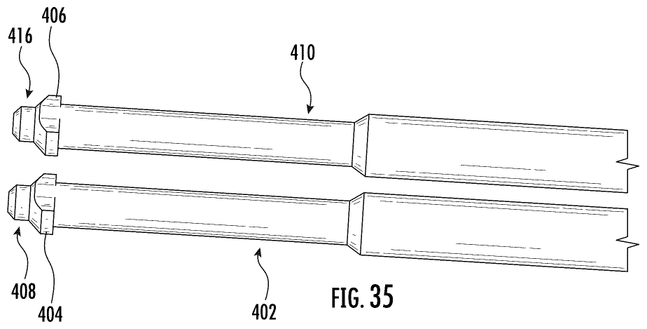

Insertion instrument 400 further includes first and second actuator shafts 402, 410 extending from the handle 430 to the bores 80, 152 within the height and angle translation members 16, 18, respectively. First and second shafts 402, 410 are positioned on opposite lateral portions of the instrument and are both attached to rotatable knobs 440, 442 on the proximal handle for longitudinally translating the shafts. As shown in FIG. 35, each shaft 402, 410 includes a distal tapered end 408, 416 and at least two projections 404, 406 extending laterally outward from the shaft proximal of the distal tapered end. The two projections 404, 406 are designed to translate through a proximal opening of the bores 80, 152 on the height and angle translation members 16, 18. Rotation of the shafts 402, 410 causes the two projections 404, 406 to slide into the two recesses within these bores, thereby locking the shafts to the height and angle translation members. Rotation of the knobs 440, 442 on the handle then causes the shafts and the translation members to move longitudinally.

The distal tapered ends 408, 416 of the instrument shafts 402, 410 are designed to engage the internal projections within the bores of the angle and height translation members. Proximal movement of the instrument shafts causes the distal tapered end to engage these projections and urge the elongate distal members of the bores laterally outward to allow reverse or distal movement of the angle and translation members.

The proximal handle 430 may also comprise position indicators (not shown) configured to indicate the position of each actuator shaft 402, 410, respectively. The position indicators may comprise visual markings or the like.

The handle further includes a third rotatable knob 444 coupled to the shaft for rotating the shaft and the endplate therewith relative to the handle. This allows for rotation of the endplate without rotating the handle to facilitate ease of use during implantation.

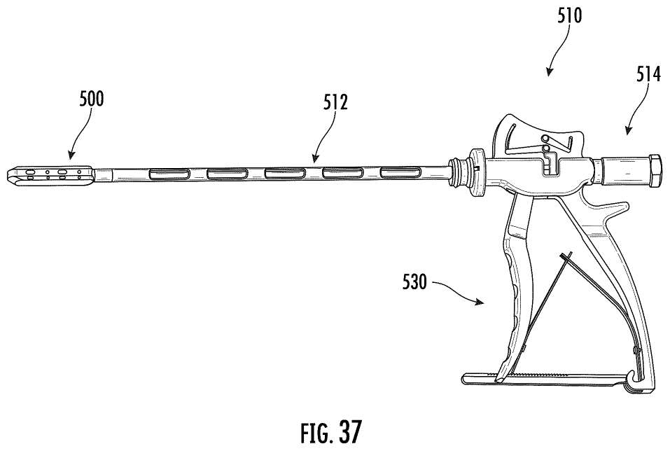

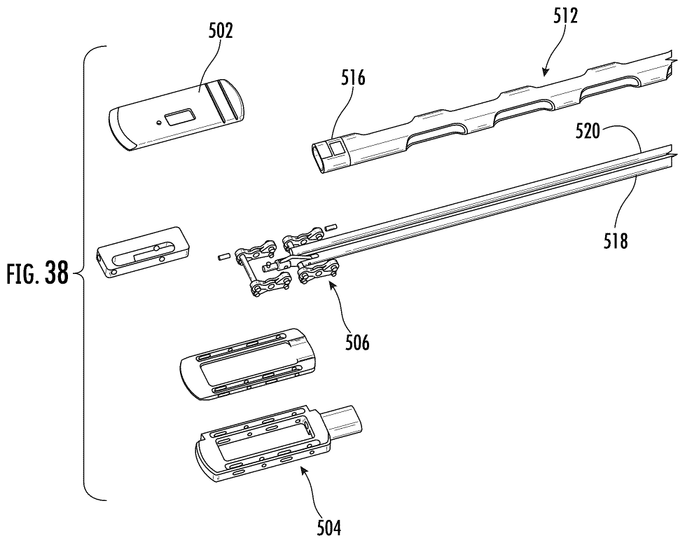

Referring now to FIGS. 37-39, a trial implant 500 and an instrument 510 for inserting implant 500 between two adjacent vertebral bodies will now be described. Trial implant 500 may be useful for determining an optimal size and shape that corresponds with the size and shape of the space between adjacent vertebral bodies of a particular patient. Trial implant 500 will typically be inserted between the vertebral bodies and adjusted in height and angle until the physician determines the optimal desired size of the spinal implant. Trial implant 500 will then be removed so that the physician can select the proper spinal implant for the procedure.

As shown, trial implant 500 generally comprises first and second endplates 502, 504 and an articulation assembly 506 for adjusting the height and angle of implant 500. Instrument 510 generally includes an elongate shaft 512, a proximal handle 514 and a distal end 516 coupled to lower endplate 504. Instrument 510 further comprises first and second actuator shafts 518, 520 extending through shaft 512. Articulation assembly 506 of trial implant 500 is coupled to first and second actuator shafts 518, 520 for adjusting the height and angle of implant 500.

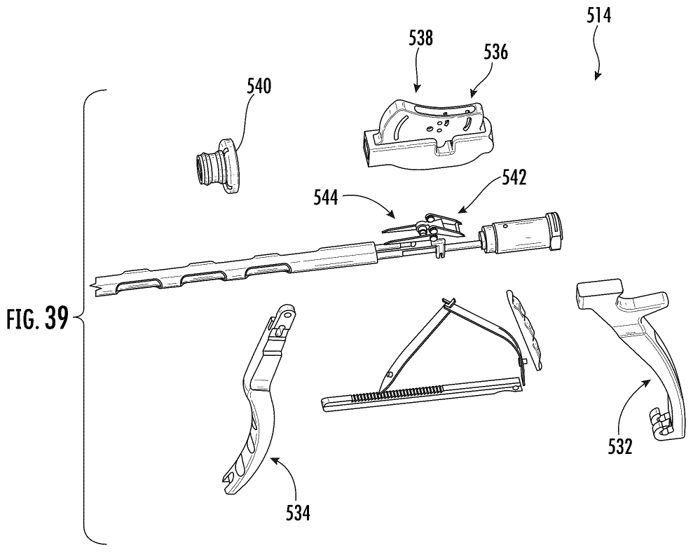

As shown in FIGS. 37 and 39, proximal handle 514 comprises a grip 530 that includes proximal and distal arms 532, 534. Proximal arm 532 is coupled to first actuator shaft 518 and distal arm 534 is coupled to second actuator shaft 520 to move these shafts longitudinally to adjust the height and angle, respectively, of trial implant 500. Proximal handle 514 further includes visual indicators 536, 538 that provide markings to illustrate the height and angle adjustments. Proximal and distal arms 532, 534 are each coupled to linkages 542, 544 that rotate relative to visual indicators 536, 538 to provide specific height (i.e., distance between the endplates) and lordosis angle (i.e., degrees of angle between endplates) indications to the user. Proximal handle 514 may also include a rotatable knob 540 for rotating the shaft and the endplate therewith relative to the handle.

The entire implant is fabricated through additive manufacturing techniques, such as 3D printing. The implant is formed layer by layer in the longitudinal direction from the proximal end to the distal end. Upon completion of manufacturing, the upper and lower endplates are coupled to each other at certain locations and then separated by a wire EDM process that creates linear cuts to separate the endplates. The hinge rod is machined separately and then inserted through openings in the proximal and distal ends of the lower endplate and through the cylinders in the upper and lower portions of the height translation member.

In an exemplary embodiment, the implants are produced by Selective Laser Melting (SLM). For example, a substrate plate is fastened to an indexing table inside a chamber with a controlled atmosphere of inert gas (e.g., argon or nitrogen). Metal powder is applied flat to the substrate plate as a layer. The metal powder is preferably a titanium alloy, e.g. Ti-6Al-4V to enable biocompatibility. Each 2D slice of the cage is fused by selectively melting the metal powder via a laser. The laser has enough energy to fully melt or rather weld the metal particles to form solid metal. The substrate plate is lowered by the layer thickness (z-direction). New metal powder is applied and the process is repeated layer by layer until the part is complete. The completed part is removed from the substrate plate by cutting or breaking off.

Preferably, all components of the cage are printed nested within each other. Compared to separately 3D printing all components next to each other, a higher utilization rate can be achieved. This means that during 3D printing, a higher proportion which is melted and a lower proportion which stays as metal powder can be achieved. Thus, production time and costs can be reduced significantly.

After 3D printing, areas connecting single components of the cage are cut by electrical discharge machining (EDM) to enable their separate movement. Further, EDM can be used to realize smooth surfaces, e.g., to enable low-friction sliding of two components against each other. With EDM, the cage can also be removed from the substrate plate.

To lower production costs, several cages can be printed onto one substrate plate. In this case, before removing the cages, EDM can be used to simultaneously cut all cages placed on the substrate plate.

The implant may comprise one or more exhaust openings in the upper and lower endplates to allow for extraction of the metal powder remaining in the cage after 3D printing. Preferably, the exhaust opening is positioned on a lateral surface of the moving plate. It is also possible to position the exhaust opening on a horizontal surface of the cage, preferably on the base plate or on the moving plate. Preferably, the cage comprises multiple exhaust openings. Thus, more areas inside the cage are reachable and the metal powder can be extracted more efficiently. It is also possible to configure an external sliding means, preferably a conical groove, in such a way that it can be additionally used as an exhaust opening. Therefore, the conical groove is deepened until a passage to the outside has been made.

Other embodiments will be apparent to those skilled in the art from consideration of the specification and practice of the embodiment disclosed herein. It is intended that the specification and examples be considered as exemplary only, with a true scope and spirit of the embodiment being indicated by the following claims.

What is claimed is:

1. An adjustable spinal fusion device, comprising:
an upper plate having an outer surface for placement against a first vertebral body, proximal and distal ends, and first and second sides extending from the proximal end to the distal end;
a lower plate having an outer surface for placement against a second vertebral body, proximal and distal ends, and first and second sides extending from the proximal end to the distal end;
first and second translation members configured to move longitudinally relative to the upper and lower plates to move the first and second sides of the upper plate away from the first and second sides of the lower plate; and
wherein the second translation member is configured to move longitudinally relative to the first translation member to move the first side of the upper plate away from the first side of the lower plate.

2. The adjustable spinal fusion device of claim 1, wherein the second translation member includes a movable wedge with at least one angled surface and the first translation member includes at least one angle surface positioned to cooperate with the movable wedge of the second translation member.

3. The adjustable spinal fusion device of claim 2, wherein the movable wedge and the at least one angled surface are positioned on the first side of the upper and lower plates such that longitudinal movement of the second translation member causes the first side of the upper plate to move towards and away from the first side of the lower plate.

4. The adjustable spinal fusion device of claim 1, further comprising a hinge on one of the first and second sides for rotatably coupling the upper plate to the lower plate.

5. The adjustable spinal fusion device of claim 1, wherein the first translation member comprises a first bore with a mating feature for cooperating with a first actuator shaft of a surgical instrument to move the first translation member in the longitudinal direction.

6. The adjustable spinal fusion device of claim 5, wherein the second translation member comprises a second bore with a mating feature for cooperating with a second actuator shaft of a surgical instrument to move the second translation member in the longitudinal direction.

7. The adjustable spinal fusion device of claim 6, wherein the first and second bores each include at least one recess formed therein for receiving a projection on the first and second actuator shafts to secure the first actuator shaft to the first translation member and the second actuator shaft to the second translation member, wherein each of the elongate members of the first and second bores are configured to mate with a tapered distal end of the first and second actuator shafts of an instrument, wherein distal movement of the first and second actuator shafts causes the tapered distal end to engage the projections and urge the fingers laterally outward.

8. The adjustable spinal fusion device of claim 7, further comprising first and second ratchet shafts extending into a distal portion of the first and second bores and having external projections configured to provide incremental adjustments of a distance and an angle between the upper and lower plates.

9. A spinal fusion system, comprising:
an adjustable spinal fusion device comprising:
an upper plate having an outer surface for placement against a first vertebral body, proximal and distal ends, and first and second sides extending from the proximal end to the distal end;
a lower plate having an outer surface for placement against a second vertebral body, proximal and distal ends, and first and second sides extending from the proximal end to the distal end; and
first and second translation members; and
an instrument having a proximal handle, an elongate shaft and an actuator within the elongate shaft coupled to the proximal handle, wherein the actuator comprises a first interface configured to move the first and second translation members relative to the upper and lower plates to move the first and second sides of the upper plate away from the first and second sides of the lower plate and a second interface configured to move the second translation member relative to the first translation member to move the first side of the upper plate away from the first side of the lower plate.

10. The spinal fusion system of claim 9, wherein the actuator comprises a first rotatable shaft coupled to the first translation member and a second rotatable shaft coupled to the second translation member.

11. The spinal fusion system of claim 10, wherein the first translation member comprises a first bore with a mating feature for cooperating with the first rotatable actuator shaft and the second translation member comprises a second bore with a mating feature for cooperating with the second rotatable actuator shaft.

12. The spinal fusion system of claim 11, wherein the first and second first actuator shafts are rotatable within the first and second bores, respectively, between a locked position, wherein the first and second first actuator shafts are coupled to the first and second translation members and an unlocked position wherein the first and second first actuator shafts are decoupled from the first and second translation members.

13. The spinal fusion system of claim 12, wherein longitudinal movement of the first and second first actuator shafts causes longitudinal movement of the first and second translation members when the first and second first actuator shafts are in the locked position.

14. The spinal fusion system of claim 9, wherein the proximal handle comprises first and second position indicators coupled to the first and second first actuator shafts and configured to provide a visual indication of a longitudinal position of the first and second first actuator shafts relative to the upper and lower plates of the adjustable spinal fusion device.

15. A method for implanting a spinal fusion device, the method comprising:
providing a spinal fusion device with first and second translation members, an upper plate having proximal and distal ends, and first and second sides extending from the proximal end to the distal end and a lower plate having proximal and distal ends, and first and second sides extending from the proximal end to the distal end;
advancing the spinal fusion device into an intervertebral space with an instrument;
actuating a first interface on the instrument to move the first and second translation members longitudinally relative to the upper and lower plates to move the first and second sides of the upper plate away from the first and second sides of the lower plate; and
actuating a second interface on the instrument to move the second translation member longitudinally relative to the first translation member to move the first side of the upper plate away from the first side of the lower plate.

16. The method of claim 15, further comprising translating first and second actuator shafts through first and second bores of the first and second translation members.

17. The method of claim 16, further comprising rotating the first and second actuator shafts relative to the first and second translation members to attach the first and second actuator shafts to the first and second translation members.

18. The method of claim 17, further comprising translating the first and second actuator shafts longitudinally relative to the upper and lower plates to translate the first and second translation members longitudinally relative to the upper and lower plates and move the first and second sides of the upper plate away from the first and second sides of the lower plate.

19. The method of claim 17, further comprising translating the second actuator shaft relative to the first actuator shaft to move the second translation member longitudinally relative to the first translation member and move the first side of the upper plate away from the first side of the lower plate.

20. The method of claim 16, further comprising rotating first and second knobs on a proximal handle of the instrument to move the first and second translation members longitudinally relative to the upper and lower plates.
